SEQUENCE LISTING (7 sequences)

SEQ ID NO: 1 - DNA, 2663 bp, guinea pig adenovirus
  misc_feature (24)..(24): n represent any nucleic acid
atggcgacgccgtcgatgatgccncagtggtcgtacatgcacatcgccggccaggaggcggtagactacctgtctcccggcctggtgcaattcgcccgcgccactgacagttatttccatctgggcaataagtttcgtaaccccaccgtggcacccactcaagaggtcacgaccgaccgctctcagagactccaactgcggtttgtgcctgtggatcgggaggacactcagtatgcgtataagacccgttttcaactggccgtgggagacaatcgcgtcttggacatgggcagcacgtacttcgacattcgcggcaccatcgaccgcgggccctccttcaaaccctacagcggcacggcctacaacagtctggcgcccaagggcgcgtccaacaacaccatgtacacccacgtgaacaaccagcagcagcaggtcggcgtggtggcacaggcggcctttttgacggaaaacatcgacccccagaacggtatacaggtccgcgtggacgccaacggtcaggccgtacgcgctcaggcgcgtttcgaacccgaacccaacgtcggtaacgagacttgggtgtatcacgacacggtgcagcgcgacgtgggtcccgtggccggacgtgtgttgaaaggcgacgtcatgcccatgccctgctacggctcctacgcccgccccaccggtgccgacggcggtcagtcggtggacaaccagatagatctcaccctgctgcgtagcggcaacgccgcgggcgcgcccgagatcgccctgtacgccgaaaacgtggacctggagactcccgacacgcatctggtgtctcgcgtgggtcccggcgaggctcgcctggccccggcgctgggacagatagcgcagcccaaccgacccaattacgtagccttccgcgacaacttcatcggactgatgtactacaacagcagcggaaacctgggcgtgctggccggtcagtcgtcgcagctcaacgcggtggtggagctgcaggaccgcaacaccgagctgtcctaccagctgctgttagacagcctggtggatcgcacgcggtattttgccatgtggaatcaggccgtggacagctacgacccccacgttcgcgtcattgagaatcacggcgtggaagacgaaatgccgaattactgttttcccctttcgggcatggtgttggaagaggccacgcaggtggacgcgcaaaacgcccgggttaacaaccaggggccgtcctacgccggcgtgggcaacgttcaggccatggagataaatctgactcagaacctgtggcgagggttcctgtactccaacgtggccctgtacctgcccgacaacctcaagttcactccgcgtaacatcattttgcccgaaaaccgcaacacctacgcttacatcaacgggcgactgccacccagcggcatcgtggacggctacatcgacatcggcgcccgctggtctcccgacgtcatggactccgtcaacccgttcaaccaccatcgcaacacgggtctgcgctatcggtctcagctgctgggcaacggccgctacgccgtgttccacatacaggtgccccaaaagtttttcgccattcgcaatctgctgctgctgccgggcacatacacctacgagtggtccttccgcaaagacgtgaacatggtgctgcaaagcaccctgggaaacgatctgcgggcggacggcgcctccatcaacatcgataacgtgaccctgtacgccagcttttttccactggcacacagcacggccgccaccctggagctcatgctacgtaacgagaccaacgatcagagcttcaacgattacctctcggccgccaacatgctgtaccccatccccgccggggccaccacggtgcccatctccattccgtgtcgtaactgggcgggcttccgcggctggagcttcgcccgtctaaaacaacgcgaaacgccctcgctgggatcccctttcgacccctactttacgtactcaggtaccataccctacctggacggcactttctacctcaaccacacgttccgtcgagtcgccatacagttcgactcgtcggtcagctggccgggcaacgaccgcctgctgtcgcccaacgagttcgaaatcaagcggtacgtggacggcgagggttacaacatcggcccgagcaacatgaccaaggactggttcctggttcagatgctggcgcactacaacatcggctatcagggctaccatctgcccgaaaacttcaaagacaggctgtattcgtttctcagaaactttcagcccctgtgtagacagataccggatccctcccatcccaattatcgcaacgttccctttacccgtcagcacgactcctcgggttttacatcccacaacctggccgtgggcgtgccggaaggtcacccctatccggccaactggccgtacccgctaatcggcgctcgggccgtgagaactttgacccaaaaaaagtttttggtggaccgtacgctgtggcgcattccctcttccagcaactttatgagtatgggagcgctgaccgattatggacaaaacctgttatacgccaattgcggtcacgctttagacatgacttttgaagtagaccccctggacgaggctaccgtattgtacgttttatttgaagtgttcgacgtggt

SEQ ID NO: 2 - protein, 875 aa, guinea pig adenovirus
MHIAGQEAVDYLSPGLVQFARATDSYFHLGNKFRNPTVAPTQEVTTDRSQRLQLRFVPVDREDTQYAYKTRFQLAVGDNRVLDMGSTYFDIRGTIDRGPSFKPYSGTAYNSLAPKGASNNTMYTHVNNQQQQVGVVAQAAFLTENIDPQNGIQVRVDANGQAVRAQARFEPEPNVGNETWVYHDTVQRDVGPVAGRVLKGDVMPMPCYGSYARPTGADGGQSVDNQIDLTLLRSGNAAGAPEIALYAENVDLETPDTHLVSRVGPGEARLAPALGQIAQPNRPNYVAFRDNFIGLMYYNSSGNLGVLAGQSSQLNAVVELQDRNTELSYQLLLDSLVDRTRYFAMWNQAVDSYDPHVRVIENHGVEDEMPNYCFPLSGMVLEEATQVDAQNARVNNQGPSYAGVGNVQAMEINLTQNLWRGFLYSNVALYLPDNLKFTPRNIILPENRNTYAYINGRLPPSGIVDGYIDIGARWSPDVMDSVNPFNHHRNTGLRYRSQLLGNGRYAVFHIQVPQKFFAIRNLLLLPGTYTYEWSFRKDVNMVLQSTLGNDLRADGASINIDNVTLYASFFPLAHSTAATLELMLRNETNDQSFNDYLSAANMLYPIPAGATTVPISIPCRNWAGFRGWSFARLKQRETPSLGSPFDPYFTYSGTIPYLDGTFYLNHTFRRVAIQFDSSVSWPGNDRLLSPNEFEIKRYVDGEGYNIGPSNMTKDWFLVQMLAHYNIGYQGYHLPENFKDRLYSFLRNFQPLCRQIPDPSHPNYRNVPFTRQHDSSGFTSHNLAVGVPEGHPYPANWPYPLIGARAVRTLTQKKFLVDRTLWRIPSSSNFMSMGALTDYGQNLLYANCGHALDMTFEVDPLDEATVLYVLFEVFDV

SEQ ID NO: 3 - DNA, 24 bp, Artificial Sequence
  5′ primer for cloning GPAdV
atggcbacsccbtcgatgmtggcc

SEQ ID NO: 4 - DNA, 26 bp, Artificial Sequence
  Derived consensus sequence for the 3′end of GPAdV
accacgtcraaracttcaaayaaaac

SEQ ID NO: 5 - DNA, 2349 bp, guinea pig adenovirus
caccatcgaccgcgggccctccttcaaaccctacagcggcacggcctacaacagtctggcgcccaagggcgcgtccaacaacaccatgtacacccacgtgaacaaccagcagcagcaggtcggcgtggtggcacaggcggcctttttgacggaaaacatcgacccccagaacggtatacaggtccgcgtggacgccaacggtcaggccgtacgcgctcaggcgcgtttcgaacccgaacccaacgtcggtaacgagacttgggtgtatcacgacacggtgcagcgcgacgtgggtcccgtggccggacgtgtgttgaaaggcgacgtcatgcccatgccctgctacggctcctacgcccgccccaccggtgccgacggcggtcagtcggtggacaaccagatagatctcaccctgctgcgtagcggcaacgccgcgggcgcgcccgagatcgccctgtacgccgaaaacgtggacctggagactcccgacacgcatctggtgtctcgcgtgggtcccggcgaggctcgcctggccccggcgctgggacagatagcgcagcccaaccgacccaattacgtagccttccgcgacaacttcatcggactgatgtactacaacagcagcggaaacctgggcgtgctggccggtcagtcgtcgcagctcaacgcggtggtggagctgcaggaccgcaacaccgagctgtcctaccagctgctgttagacagcctggtggatcgcacgcggtattttgccatgtggaatcaggccgtggacagctacgacccccacgttcgcgtcattgagaatcacggcgtggaagacgaaatgccgaattactgttttcccctttcgggcatggtgttggaagaggccacgcaggtggacgcgcaaaacgcccgggttaacaaccaggggccgtcctacgccggcgtgggcaacgttcaggccatggagataaatctgactcagaacctgtggcgagggttcctgtactccaacgtggccctgtacctgcccgacaacctcaagttcactccgcgtaacatcattttgcccgaaaaccgcaacacctacgcttacatcaacgggcgactgccacccagcggcatcgtggacggctacatcgacatcggcgcccgctggtctcccgacgtcatggactccgtcaacccgttcaaccaccatcgcaacacgggtctgcgctatcggtctcagctgctgggcaacggccgctacgccgtgttccacatacaggtgccccaaaagtttttcgccattcgcaatctgctgctgctgccgggcacatacacctacgagtggtccttccgcaaagacgtgaacatggtgctgcaaagcaccctgggaaacgatctgcgggcggacggcgcctccatcaacatcgataacgtgaccctgtacgccagcttttttccactggcacacagcacggccgccaccctggagctcatgctacgtaacgagaccaacgatcagagcttcaacgattacctctcggccgccaacatgctgtaccccatccccgccggggccaccacggtgcccatctccattccgtgtcgtaactgggcgggcttccgcggctggagcttcgcccgtctaaaacaacgcgaaacgccctcgctgggatcccctttcgacccctactttacgtactcaggtaccataccctacctggacggcactttctacctcaaccacacgttccgtcgagtcgccatacagttcgactcgtcggtcagctggccgggcaacgaccgcctgctgtcgcccaacgagttcgaaatcaagcggtacgtggacggcgagggttacaacatcggcccgagcaacatgaccaaggactggttcctggttcagatgctggcgcactacaacatcggctatcagggctaccatctgcccgaaaacttcaaagacaggctgtattcgtttctcagaaactttcagcccctgtgtagacagataccggatccctcccatcccaattatcgcaacgttccctttacccgtcagcacgactcctcgggttttacatcccacaacctggccgtgggcgtgccggaaggtcacccctatccggccaactggccgtacccgctaatcggcgctcgggccgtgagaactttgacccaaaaaaagtttttggtggaccgtacgctgtggcgcattccctcttccagcaactttatgagtatgggagcgctgaccgattatggacaaaacctgttatacgccaattgcggtcacgctttagacatgacttttgaagtagaccccctggacgaggctaccgtattgtacgttttatttgaagtgttcgacgtggt

SEQ ID NO: 6 - protein, 769 aa, guinea pig adenovirus
TAYNSLAPKGASNNTMYTHVNNQQQQVGVVAQAAFLTENIDPQNGIQVRVDANGQAVRAQARFEPEPNVGNETWVYHDTVQRDVGPVAGRVLKGDVMPMPCYGSYARPTGADGGQSVDNQIDLTLLRSGNAAGAPEIALYAENVDLETPDTHLVSRVGPGEARLAPALGQIAQPNRPNYVAFRDNFIGLMYYNSSGNLGVLAGQSSQLNAVVELQDRNTELSYQLLLDSLVDRTRYFAMWNQAVDSYDPHVRVIENHGVEDEMPNYCFPLSGMVLEEATQVDAQNARVNNQGPSYAGVGNVQAMEINLTQNLWRGFLYSNVALYLPDNLKFTPRNIILPENRNTYAYINGRLPPSGIVDGYIDIGARWSPDVMDSVNPFNHHRNTGLRYRSQLLGNGRYAVFHIQVPQKFFAIRNLLLLPGTYTYEWSFRKDVNMVLQSTLGNDLRADGASINIDNVTLYASFFPLAHSTAATLELMLRNETNDQSFNDYLSAANMLYPIPAGATTVPISIPCRNWAGFRGWSFARLKQRETPSLGSPFDPYFTYSGTIPYLDGTFYLNHTFRRVAIQFDSSVSWPGNDRLLSPNEFEIKRYVDGEGYNIGPSNMTKDWFLVQMLAHYNIGYQGYHLPENFKDRLYSFLRNFQPLCRQIPDPSHPNYRNVPFTRQHDSSGFTSHNLAVGVPEGHPYPANWPYPLIGARAVRTLTQKKFLVDRTLWRIPSSSNFMSMGALTDYGQNLLYANCGHALDMTFEVDPLDEATVLYVLFEVFDV

SEQ ID NO: 7 - DNA, 80 bp, guinea pig adenovirus
ctgtacctgcccgacaacctcaagttcactccgcgtaacatcattttgcccgaaaaccgcaacacctacgcttacatcaa

(19) United States
(12) Patent Application Publication
(10) Pub. No.: US 2002/0123040 A1
(43) Pub. Date: Sep. 5, 2002

(54) Novel guinea pig adenovirus antigen

(76) Inventors: Sanford H. Feldman, Ruckersville, VA (US); Nicholas Douris, Charlottesville, VA (US)

Correspondence Address: John Breen, University of Virginia Patent Foundation, Suite 1-110, 1224 West Main Street, Charlottesville, VA, 22903, US

(21) Appl. No.: 10/087,110

(22) Filed: Mar. 1, 2002

Related U.S. Application Data
(60) Provisional application No. 60/272,443, filed on Mar. 1, 2001.

(51) Int. Cl. C12Q 1/70; C07H 21/04; C12N 9/00; A61K 39/12

(52) U.S. Cl. 435/5; 435/183; 424/204.1; 536/23.72; 530/388.3

(57) ABSTRACT

The present invention relates to the isolation of the Guinea Pig Adenovirus (GPAdV) Hexon gene and the determination of the encoded polypeptide. The purified GPAdV hexon coat protein, or bioactive fragments thereof, can be used in accordance with one embodiment to detect and study the incidence and/or development of antibodies in guinea pigs exposed to GPAdV.

### FIELD OF THE INVENTION

The present invention is directed to the Guinea Pig Adenovirus (GPAdV) Hexon coat protein and to nucleic acid sequences encoding that protein. This antigenic protein can be used to prepare vaccine formulation for the prevention of disease in guinea pigs.

### BACKGROUND OF THE INVENTION

Adenovirus as an infectious cause of bronchial pneumonia in guinea pigs was first reported in Germany and later in North America, Australia and several European countries. Initial descriptions of this disease related to lethal adenoviral pneumonia of guinea pigs. The presence of subclinical disease with this virus was suspected in these reports, however supportive serologic and histological evidence was lacking. Kunstyr et al. Lab Anim. 18:55-60 (1984) was the first to reproduce the disease in newborn guinea pigs. To date no in vitro method has been found supporting propagation of this virus sufficient to allow isolation of genomic DNA for molecular biological analysis. Antigens derived from other mastadenoviruses of animals and humans have failed to reliably detect antibodies produced in guinea pigs to the presence of this virus.

A polymerase chain reaction (PCR) was developed to a 281 base pair (bp) portion of the gene encoding the hexon coat protein of the guinea pig adenovirus. An abbreviated form of this PCR was incorporated with histological and serologic methods to study the pathogenesis of the guinea pig adenovirus in the respiratory tract (nasal turbinate mucosa) of guinea pigs. This latter study documented that the life cycle of this virus appeared to be transient replication in the upper respiratory mucosa for up to 15 days causing an “occasional sneeze” with no observed signs of clinical or lethal pneumonia. Little else is known about guinea pig adenovirus (GPAdV) in relation to other adenoviruses in the genus mastadenovirus.

The present invention is directed to the isolation, cloning and sequencing of approximately 98% of the gene encoding the hexon coat protein of the GPAdV from preserved tissue obtained during an outbreak of this disease. The GPAdV nucleic acid sequence was used to deduce the amino acid sequence of the cloned portion of the hexon coat protein gene. The present invention represents the first describe amino acid sequence for the GPAdV hexon coat protein.

### SUMMARY OF THE INVENTION

The present invention is directed to the Guinea Pig Adenovirus Hexon coat protein and to nucleic acid sequences encoding that protein. This antigenic protein can be used to prepare vaccine formulation for the prevention of disease in guinea pigs. The present invention is also directed to an assay for detecting the presence of anti-GPAdV hexon coat antibodies in individual animals. The assay utilizes the hexon polypeptide of SEQ ID NO: 2, or a fragment thereof and a labeled secondary antibody to detect the presence of GPAdV antibodies in sample recovered from the animal to be tested. Importantly this assay can be used to monitor exposure of the animal to the virus and thus prevent the spread of the disease.

### BRIEF DESCRIPTION OF THE DRAWINGS

FIG. 1 depicts the PRETTY amino acid sequence alignment deduced for the hexon clone (GPAdV hexo) and human adenovirus types 2 (human ad 2, SWISSPRO accession HEX_ADE02) and 5 (human ad 5, SWISSPRO accession HEX_ADE05). The human adenovirus hexon sequences are truncated at the carboxy-terminal to coincide with the end of the deduced sequence of the GPAdV hexon fragment cloned. Where the three sequences are in consensus an upper case letter is used in the consensus line and (-) where there is no consensus. For the individual amino acid sequences, the symbol legend (˜) is where no input sequence exists, (-) is where there is agreement of individual sequences with the consensus line, and (•) where a gap is made in the individual sequence to facilitate the alignment procedure. Lower case letters are shown in the individual amino acid sequences where there is no consensus. The shaded bars indicate the topological regions that correspond with the amino acid sequence according to references Athappilly et al., J. Mol. Biol. 242:430-455 (1994) and Rux et al., Molecular Therapy 1:18-30 (2000).

### DETAILED DESCRIPTION OF THE INVENTION

Definitions

In describing and claiming the invention, the following terminology will be used in accordance with the definitions set forth below.

As used herein, “nucleic acid,” “DNA,” and similar terms also include nucleic acid analogs, i.e. analogs having other than a phosphodiester backbone. For example, the so-called “peptide nucleic acids,” which are known in the art and have peptide bonds instead of phosphodiester bonds in the backbone, are considered within the scope of the present invention.

The term “peptide” encompasses a sequence of 3 or more amino acids wherein the amino acids are naturally occurring or synthetic (non-naturally occurring) amino acids. Peptide mimetics include peptides having one or more of the following modifications:

1. peptides wherein one or more of the peptidyl —C(O)NR—linkages (bonds) have been replaced by a non-peptidyl linkage such as a —CH2-carbamate linkage (—CH2OC(O)NR—), a phosphonate linkage, a —CH2-sulfonamide (—CH 2—S(O)2NR—) linkage, a urea (—NHC(O)NH—) linkage, a —CH2-secondary amine linkage, or with an alkylated peptidyl linkage (—C(O)NR—) wherein R is C1-C4 alkyl;

2. peptides wherein the N-terminus is derivatized to a —NRR1 group, to a —NRC(O)R group, to a —NRC(O)OR group, to a —NRS(O)2R group, to a —NHC(O)NHR group where R and R1 are hydrogen or C1-C4 alkyl with the proviso that R and R1 are not both hydrogen;

3. peptides wherein the C terminus is derivatized to —C(O)R2 where R2 is selected from the group consisting of C1-C4 alkoxy, and —NR3R4 where R3 and R4 are independently selected from the group consisting of hydrogen and C1-C4 alkyl.

Naturally occurring amino acid residues in peptides are abbreviated as recommended by the IUPAC-IUB Biochemical Nomenclature Commission as follows: Phenylalanine is Phe or F; Leucine is Leu or L; Isoleucine is Ile or I; Methionine is Met or M; Norleucine is Nle; Valine is Val or V; Serine is Ser or S; Proline is Pro or P; Threonine is Thr or T; Alanine is Ala or A; Tyrosine is Tyr or Y; Histidine is His or H; Glutamine is Gln or Q; Asparagine is Asn or N; Lysine is Lys or K; Aspartic Acid is Asp or D; Glutamic Acid is Glu or E; Cysteine is Cys or C; Tryptophan is Trp or W; Arginine is Arg or R; Glycine is Gly or G, and X is any amino acid. Other naturally occurring amino acids include, by way of example, 4-hydroxyproline, 5-hydroxylysine, and the like.

Synthetic or non-naturally occurring amino acids refer to amino acids which do not naturally occur in vivo but which, nevertheless, can be incorporated into the peptide structures described herein. The resulting “synthetic peptide” contain amino acids other than the 20 naturally occurring, genetically encoded amino acids at one, two, or more positions of the peptides. For instance, naphthylalanine can be substituted for trytophan to facilitate synthesis. Other synthetic amino acids that can be substituted into peptides include L-hydroxypropyl, L-3,4-dihydroxyphenylalanyl, alpha-amino acids such as L-alpha-hydroxylysyl and D-alpha-methylalanyl, L-alpha.-methylalanyl, beta.-amino acids, and isoquinolyl. D amino acids and non-naturally occurring synthetic amino acids can also be incorporated into the peptides. Other derivatives include replacement of the naturally occurring side chains of the 20 genetically encoded amino acids (or any L or D amino acid) with other side chains.

As used herein, the term “conservative amino acid substitution” are defined herein as exchanges within one of the following five groups:

I. Small aliphatic, nonpolar or slightly polar residues:

Ala, Ser, Thr, Pro, Gly;

II. Polar, negatively charged residues and their amides:

Asp, Asn, Glu, Gln;

III. Polar, positively charged residues:

His, Arg, Lys;

IV. Large, aliphatic, nonpolar residues:

Met, Leu, Ile, Val, Cys

V. Large, aromatic residues:

Phe, Tyr, Trp

As used herein, the term “biologically active fragments” or “bioactive fragment” of the GPAdV hexon coat protein encompasses natural or synthetic portions of the amino acid sequence of SEQ ID NO: 2 or other fragments of the GPAdV hexon coat protein that bind to antibodies raised against the natural GPAdV hexon coat protein.

As used herein the term “solid support” relates to a solvent insoluble substrate that is capable of forming linkages (preferably covalent bonds) with soluble molecules. The support can be either biological in nature, such as, without limitation, a cell or bacteriophage particle, or synthetic, such as, without limitation, an acrylamide derivative, agarose, cellulose, nylon, silica, or magnetized particles.

A “polylinker” is a nucleic acid sequence that comprises a series of three or more different restriction endonuclease recognitions sequences closely spaced to one another (i.e. less than 10 nucleotides between each site).

As used herein, the term “vector” is used in reference to nucleic acid molecules that has the capability of replicating autonomously in a host cell, and optionally may be capable of transferring DNA segment(s) from one cell to another. Vectors can be used to introduce foreign DNA into host cells where it can be replicated (i.e., reproduced) in large quantities. Examples of vectors include plasmids, cosmids, lambda phage vectors, viral vectors (such as retroviral vectors).

As used herein a “gene” refers to the nucleic acid coding sequence as well as the regulatory elements necessary for the DNA sequence to be transcribed into messenger RNA (mRNA) and then translated into a sequence of amino acids characteristic of a specific polypeptide.

A “marker” is an atom or molecule that permits the specific detection of a molecule comprising that marker in the presence of similar molecules without such a marker. Markers include, for example radioactive isotopes, antigenic determinants, nucleic acids available for hybridization, chromophors, fluorophors, chemiluminescent molecules, electrochemically detectable molecules, molecules that provide for altered fluorescence-polarization or altered light-scattering and molecules that allow for enhanced survival of an cell or organism (i.e. a selectable marker). A reporter gene is a gene that encodes for a marker.

A promoter is a DNA sequence that directs the transcription of a DNA sequence, such as nucleotide coding sequence of a gene. Typically, a promoter is located in the 5′ region of a gene, proximal to the transcriptional start site of a structural gene. Promoters can be inducible (the rate of transcription changes in response to a specific agent), tissue specific (expressed only in some tissues), temporal specific (expressed only at certain times) or constitutive (expressed in all tissues and at a constant rate of transcription).

As used herein, the terms “complementary” or “complementarity” are used in reference to polynucleotides (i.e., a sequence of nucleotides) related by the base-pairing rules. For example, for the sequence “A-G-T,” is complementary to the sequence “T-C-A.”

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

As used herein, the term “purified” and like terms relate to the isolation of a molecule or compound in a form that is substantially free of contaminants normally associated with the molecule or compound in a native or natural environment. “Operably linked” refers to a juxtaposition wherein the components are configured so as to perform their usual function. Thus, control sequences or promoters operably linked to a coding sequence are capable of effecting the expression of the coding sequence.

As used herein, the term “pharmaceutically acceptable carrier” encompasses any of the standard pharmaceutical carriers, such as a phosphate buffered saline solution, water and emulsions such as an oil/water or water/oil emulsion, and various types of wetting agents.

As used herein, the term “treating” includes alleviating the symptoms associated with a specific disorder or condition and/or preventing or eliminating said symptoms.

The Invention

The guinea pig adenovirus has proven difficult to study due to the lack of an in vitro method of propagating the virus. Investigators have attempted to use antigens derived from other mastadenoviruses to study the incidence and/or development of antibodies in guinea pigs inoculated with GPAdV. These investigations have reported a lack of cross-reactivity, or weak cross-reactivity, of guinea pig convalescent sera when examined with heterotypic antigens.

The present invention is directed to the isolation of the GPAdV hexon gene and the determination of the encoded polypeptide. The purified GPAdV hexon coat protein, or bioactive fragments thereof, can be used in accordance with one embodiment to detect and study the incidence and/or development of antibodies in guinea pigs exposed to GPAdV.

In accordance with one embodiment a purified polypeptide is provided comprising the amino acid sequence of SEQ ID NO: 2, SEQ ID NO: 6, or an amino acid sequence that differs from the amino acid sequence of SEQ ID NO: 2 by one or more conservative amino acid substitutions. Alternatively, the polypeptide may comprise an amino acid sequence that differs from SEQ ID NO: 2 by 1 to 5 alterations, and more preferably a single alteration, wherein the alterations are independently selected from a single amino acid deletion, insertion or substitution. In one embodiment a polypeptide is provided selected from the group consisting of SEQ ID NO: 2, SEQ ID NO: 6, and antigenic fragments of SEQ ID NO: 6.

The GPAdV hexon protein of SEQ ID NO: 2 has been shown to function as an antigen marker for detecting guinea pig exposure to GPAdV. In particular, guinea pigs that have been exposed/innoculated with GPAdV contain antibodies directed against the GPAdV hexon coat polypeptide that will bind to the polypeptide of SEQ ID NO: 2, thus allowing detection of the antibodies through standard techniques. More particularly, serological testing of guinea pigs that have been exposed to GPAdV has detected anti-hexon coat proteins based on an assay using the polypeptide of SEQ ID NO: 2 and a labeled anti-guinea pig secondary antibody.

In accordance with one embodiment of the present invention, a method is provided for screening an animal for exposure to GPAdV. The method comprises the steps of obtaining an antibody containing tissue sample from the animal, preferably a blood/sera sample, and contacting the tissue sample with a hexon coat polypeptide selected from the group consisting of SEQ ID NO: 2 or an antigenic fragment of SEQ ID NO: 2 under conditions suitable to allow binding of any GPAdV antibodies present the sample to the hexon coat polypeptide. After allowing a sufficient length of time for antibodies present in the sample to bind to the hexon coat polypeptide, non-specifically bound material is removed. In one embodiment the hexon coat polypeptide is immobilized on a solid surface and the non-specifically bound material is removed by washing with buffered solutions. After the non-specifically bound material is removed, any remaining antibodies bound to the hexon coat polypeptide is detected through the use of indirect immunoassay, using a labeled secondary antibody that binds to guinea pig antibodies. The antibodies can be labeled with any of the standard markers that are typically used to detect the presence of an antibody, and include flourescent compounds, enzymes and radioisotopes.

The hexon coat polypeptide can be immobilized onto a solid support using standard techniques to allow for rapid screening. In preferred embodiments the library compounds are immobilized by linking the polypeptide to the support through a covalent bond. The solid support can be a solid surface or in particulate form, more particularly in one embodiment the hexon polypeptide is bound to a solid surface as part of a microarray. The material used as the solid surface can be selected from any surface that has been used to immobilize biological compounds including but is not limited to polystyrene, agarose, silica or nitrocellulose. In one embodiment the solid surface comprises functionalized silica or agarose.

In another aspect of the invention, the GPAdV hexon protein of SEQ ID NO: 2, or fragments thereof are delivered to a subject to elicit an active immune response. Thus in one aspect of the present invention an antigenic composition is provided, wherein said composition comprises the amino acid sequence of SEQ ID NO: 2 or an antigenic fragment of the amino acid sequence of SEQ ID NO: 2. The composition may also include any of the know pharmaceutically acceptable carriers known to those skilled in the art. Such compositions could be used for active immunization of a subject, to raise an antibody response to prevent GPAdV infection. In accordance with one embodiment a vaccine composition is provided wherein the composition comprises an amino acid sequence of SEQ ID NO: 2. The vaccines of the invention may be multivalent or univalent. Multivalent vaccines are made from recombinant viruses that direct the expression of more than one antigen.

Suitable preparations of vaccines include injectables, either as liquid solutions or suspensions; solid forms suitable for solution in, suspension in, liquid prior to injection, may also be prepared. The preparation may also be emulsified, or the polypeptides encapsulated in liposomes. The active immunogenic ingredients are often mixed with excipients which are pharmaceutically acceptable and compatible with the active ingredient. Suitable excipients are, for example, water saline, dextrose, glycerol, ethanol, or the like and combinations thereof. In addition, if desired, the vaccine preparation may also include minor amounts of auxiliary substances such as wetting or emulsifying agents, pH buffering agents, and/or adjuvants which enhance the effectiveness of the vaccine.

The present invention is also directed to antibodies that specifically bind to the GPAdV hexon protein, and more particularly, antibodies that bind to the amino acid sequence of SEQ ID NO: 2, or an antigenic fragment thereof. In accordance with one embodiment the antibody is a monoclonal antibody. These antibodies can be formulated with standard carriers and optionally labeled to prepare therapeutic or diagnostic compositions. Antibodies to the GPAdV hexon coat polypeptide are generated using methods that are well known in the art. Such antibodies may include, but are not limited to, polyclonal, monoclonal, chimeric (i.e “humanized” antibodies), single chain (recombinant), Fab fragments, and fragments produced by a Fab expression library. These antibodies can be used as diagnostic agents for the diagnosis of animal exposure to GPAdV, or in assays to monitor the success of GPAdV treatment. The antibodies useful for diagnostic purposes may be prepared in the same manner as those described above for therapeutics. The antibodies may be used with or without modification, and may be labeled by joining them, either covalently or non-covalently, with a marker.

The invention also encompasses anti-idiotypic antibodies, antagonists and agonists, as well as compounds or nucleotide constructs that inhibit expression of the GPAdV hexon protein (transcription factor inhibitors, antisense and ribozyme molecules, or gene or regulatory sequence replacement constructs), or promote expression of the GPAdV hexon protein (e.g., expression constructs in which GPAdV hexon protein coding sequences are operatively associated with expression control elements such as promoters, promoter/enhancers, etc.).

The present invention also encompasses nucleic acid sequences that encode the GPAdV hexon protein, bioactive fragments and derivatives thereof. In particular the present invention is directed to nucleic acid sequences comprising the sequence of SEQ ID NO: 1, or fragments thereof. In one embodiment, purified nucleic acids comprising at least 20 contiguous nucleotides (i.e., a hybridizable portion) that are identical to any 20 contiguous nucleotides of the nucleic acid of SEQ ID NO: 5, are provided. In other embodiments, the nucleic acids comprises at least 25 (contiguous) nucleotides, 50 nucleotides, 100 nucleotides, or 200 nucleotides of the nucleic acid of SEQ ID NO: 5. These sequences can be used as probes or PCR primers to isolate the remaining portions of the GPAdV hexon protein gene. The present invention also encompasses transgenic host cells comprising the nucleotide sequence of SEQ ID NO: 1 or SEQ ID NO: 5.

The present invention also includes nucleic acids that hybridize (under conditions defined herein) to all or a portion of the nucleotide sequence represented by SEQ ID NO: 7 or its complement. The hybridizing portion of the hybridizing nucleic acids is typically at least 15 nucleotides in length. Hybridizing nucleic acids of the type described herein can be used, for example, as a cloning probe, a primer (e.g., a PCR primer), or a diagnostic probe. It is anticipated that the DNA sequence of SEQ ID NO: 7, or fragments thereof can be used as probes to detect homologous genes from other vertebrate species.

Nucleic acid duplex or hybrid stability is expressed as the melting temperature or Tm, which is the temperature at which a nucleic acid duplex dissociates into its component single stranded DNAs. This melting temperature is used to define the required stringency conditions. Typically a 1% mismatch results in a 1° C. decrease in the Tm, and the temperature of the final wash in the hybridization reaction is reduced accordingly (for example, if two sequences having >95% identity, the final wash temperature is decreased from the Tm by 5° C.). In practice, the change in Tm can be between 0.5° C. and 1.5° C. per 1% mismatch.

The present invention is directed to the nucleic acid sequence of SEQ ID NO: 7 and purified nucleic acid sequences that hybridize to that sequence (or fragments thereof) under stringent or highly stringent conditions. In accordance with the present invention highly stringent conditions are defined as conducting the hybridization and wash conditions at no lower than −5° C. Tm. Stringent conditions are defined as involve hybridizing at 68° C. in 5× SSC/5× Denhardt's solution/1.0% SDS, and washing in 0.2× SSC/0.1% SDS at 68° C. . Moderately stringent conditions include hybridizing at 68° C. in 5× SSC/5× Denhardt's solution/1.0% SDS and washing in 3× SSC/0.1% SDS at 42° C. Additional guidance regarding such conditions is readily available in the art, for example, by Sambrook et al., 1989, Molecular Cloning, A Laboratory Manual, Cold Spring Harbor Press, N.Y.; and Ausubel et al. (eds.), 1995, Current Protocols in Molecular Biology, (John Wiley & Sons, N.Y.) at Unit 2.10.

In another embodiment of the present invention, nucleic acid sequences encoding the hexon coat polypeptide can be inserted into expression vectors and used to transfect cells to express recombinant hexon coat polypeptide in the target cells. In accordance with one embodiment, the nucleic acid sequence of SEQ ID NO: 1 are inserted into a eukaryotic expression vector in a manner that operably links the gene sequences to the appropriate regulatory sequences, and the hexon coat polypeptide is expressed in a eukaryotic host cell. Suitable eukaryotic host cells and vectors are known to those skilled in the art. In particular, nucleic acid sequences encoding the hexon coat polypeptide may be added to a cell or cells in vitro or in vivo using delivery mechanisms such as liposomes, viral based vectors, or microinjection. Accordingly, one aspect of the present invention is directed to transgenic cell lines that contain recombinant genes that express the the hexon coat polypeptide polypeptide of SEQ ID NO: 2.

In accordance with one embodiment of the present invention a mammalian expression system is used to express the cloned GPAdV truncated hexon protein of SEQ ID NO: 2. Expression vectors comprising the truncated hexon protein of SEQ ID NO: 2 as well as host cells stably or transiently transfected with such an expression vector are also encompassed by the present invention. In one aspect the expression system is used to obtain sufficient quantities of purified hexon protein to be used either in vaccine formulations or in assays to detect the presence of GPAdV antibodies in individual animals. In one preferred embodiment the presence of GPAdV in a serum or blood sample will be detected through the use of an indirect fluoresecent or enzyme linked immunosorbent assay. Such assays will allow for serologic monitoring of this pathogen in colonies of laboratory guinea pigs that should assist in the identification and eradication of this pathogenic and potentially lethal virus from laboratory colonies.

### EXAMPLE 1

Isolation of GPAdV Nucleic Acid and Protein

Materials and Methods

Isolation of the Guinea Pig Adenovirus DNA:

Deoxyribonucleic acid (DNA) of the GPAdV was isolated from a paraffin embedded histological section of infected guinea pig lung documented to contain the GPAdV in a recent epizootic. The section was deparaffinized in xylene at 50° C. for one hour. The tissue on the slide was rehydrated through graded alcohols (100%, 95% and 70%) for one hour each and placed in water for one hour. The tissue was removed from the slide with a clean razor blade and DNA isolated using a DNeasy kit (Qiagen, Valencia, Calif.) according to the manufacturer's recommendations for animal tissues. The isolated DNA was subjected to PCR using a proofreading thermostable DNA polymerase (Platinum Pfx, Life Technologies, Bethesda, Md.). DNA concentration was determined by absorbance at 260 nm. DNA purity was determined by examining the ratio of absorbances at 260 nm and 280 nm.

Development of PCR Primers:

All nucleic acid sequences for alignment and comparative analyses were obtained from either the NIH GenBank or European Molecular Biology Laboratory (EMBL) or the Swiss Protein Database (SWISSPRO). The known GPAdV hexon gene sequence (accession X95630) was aligned with that of mouse adenovirus FL strain (MAD1, accession P48308), bovine adenovirus type 3 (accession P03278) and porcine adenovirus type 3 (accession AF83132) using Omiga 1.1 software (UK). The alignment disclosed that the known sequence of the guinea pig hexon gene was near the 5′ end of the gene. A PCR primer was defined at the 5′ end beginning at the first ATG start codon for later experiments for protein expression. The sequence of this primer is ATGGCBACSCCBTCGATGMTGGCC (SEQ ID NO: 3). To identify a consensus sequence at the 3′ end, a sequence alignment was performed using hexon gene sequences from MAD1, human type 4 (accession AF065062), human type 7 (accession AF053085), human type 3 (accession S39298), human type 40 (accession P11819) and human type 41 (accession P11820). A degenerate reverse primer was identified with the consensus sequence ACCACGTCRAARACTTCAAAYAAAAC (SEQ ID NO: 4).

High Fidelity PCR:

100 ng of the isolated DNA was subjected to a polymerase chain reaction (PCR) using a master mix containing 1 unit of Pix polymerase, 1× Platinum Pfx buffer, 0.5μM of each primer species, 200 mM of each nucleotide triphosphate and 1.5 mM MgSO₄. The amplification was performed on a Robocycler 9600 thermalcycler (Stratagene, La Jolla, Calif.) set for 40 cycles with the following parameters: 94° C. for 45 seconds, 60° C. for one minute, 68° C. for 2.5 minutes. This was followed by a final extension at 68° C. for ten minutes for one cycle. The reaction product was analyzed by 1% agarose (Life Technologies, Bethesda, Md.) gel electrophoresis in 1× Tris acetate EDTA buffer containing 0.5μg/ml ethidium bromide run at 90 volts for 30 minutes and the results photographed.

Cloning of PCR Product:

The PCR product was purified from the agarose gel using a Gel Extraction kit (Qiagen, Valencia, Calif.) according to manufacturer's recommendations. The isolated fragment was cloned into pCR-Blunt II TOPO (Invitrogen, Carlsbad, Calif.) and transfected into chemically competent Top 10 E. coli (Invitrogen, Carlsbad, Calif.) according to the manufacturer's instructions. Recombinant clones were selected on Luria Bertani (LB) agar (Life Technologies, Bethesda, Md.) containing kanamycin (50 μg/ml). Recombinant clones were identified by increased molecular size compared to the parent plasmid when analyzed by agarose gel electrophoresis. Clones were further analyzed by Pvu II restriction digest (NEBiolabs, Beverly, Mass.) to identify the orientation of the insert (data not shown). Plasmid preparations of two clones in opposite orientations were grown in LB broth containing kanamycin (50 μg/ml). Both plasmids were isolated using Plasmid mini-kit (Qiagen, Valencia, Calif.). The purity and concentration of the plasmids was validated by UV spectrophotometry (see GPAdV DNA isolation). These plasmids were submitted to the University of Virginia Biomolecular Research Facility for nucleic acid sequence determination, and the sequences of the two plasmids were compared by PRETTY (GCG/Oxford Molecular Group, UK) sequence alignment.

Nucleic Acid and Amino Acid Sequence Analysis:

The cloned DNA sequence was analyzed using various programs within the SeqWeb package of computer analysis software (GCG/Oxford Molecular Group, UK). Initially the nucleic acid sequence was aligned with GenBank accession X95630 to confirm the identity of the cloned fragment using the PRETTY software (GCG/Oxford Molecular Group, UK). The amino acid sequence of the cloned fragment was deduced using TRANSLATE software (GCG/Oxford Molecular Group, UK). The deduced amino acid sequence was subjected to a BLAST search using the NIH National Center for Biotechnology Information (NCBI) World Wide Web application, the results represented using TAXBLAST option to obtain GenBank, and SWISSPRO protein accessions with similar sequences of amino acids. The adenovirus hexon peptide sequences identified by the TAXBLAST search were imported and truncated to align with the C-terminal end of the deduced GPAdV amino acid sequence for further analysis.

To identify structural regions defined by the deduced amino acid sequence of the GPAdV hexon, a comparison was made with human adenovirus types 2 and 5. The hexon sequences of human adenovirus types 2 and 5 were truncated to the size of the deduced GPAdV amino acid sequence and the three subjected to a multiple sequence alignment using the PRETTY software (GCG/Oxford Molecular Group, UK) for presentation with a consensus sequence.

To determine the similarity of the deduced amino acid sequence of GPAdV hexon to that of other mastadenoviruses a similarity matrix was established. Hexon amino acid sequences from GPAdV and 25 mastadenoviruses identified by the TAXBLAST search (NCBI) were analyzed using the DISTANCES software (GCG/Oxford Molecular Group, UK) incorporating the Kimura Protein Method option. With the Kimura option the similarity score is related only to the number of exact matches and gap positions are ignored. This similarity matrix was further analyzed with the GROWTREE software (GCG/Oxford Molecular Group, UK) to produce a schematic tree-like schematic representation of mastadenovirus relatedness by comparing small peptide regions for similarity and using the neighbor joining method for minimization of overall branch length.

Results

Three overlapping amplification products were cloned and sequenced that identified 2663 nucleic acids in a single open reading frame. The deduced amino acid sequence of the cloned fragment consisted of a single open reading frame (+1) of 887 amino acids beginning with the ATG codon in the 5′ PCR primer and spanning the entire cloned fragment. At the two positions where the cloned nucleotide sequence differed from GenBank accession X95630, the deduced amino acid sequence did not differ. In GenBank accession X95630 nucleotide positions 70-72 ACC encode threonine as does the cloned nucleotide sequence ACT. Similarly, GenBank accession X95630 nucleotide positions 268-270 TTT encode phenylalanine as do the clone's nucleotide sequence of TTC.

The Dayhoff matrix of similarity demonstrated the GPAdV hexon amino acid sequence to be less than 80% similar to those of other adenovirus hexons studied. The GPAdV amino acid sequence was most similar to that of canine adenoviruses type 1 (68%) least similar to that of bovine type 8 (28%). The hexon of mouse adenovirus type 1 (MAD-1), the virus most often used as antigen for detection of antibodies to GPAdV in guinea pigs, has 54% similarity to the GPAdV hexon.

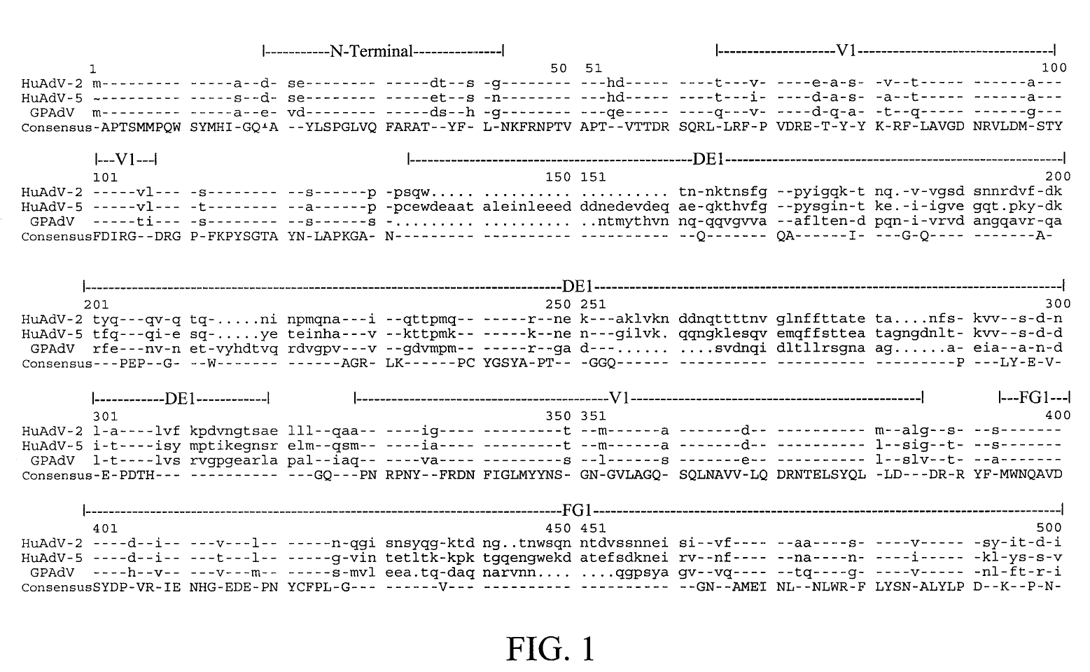

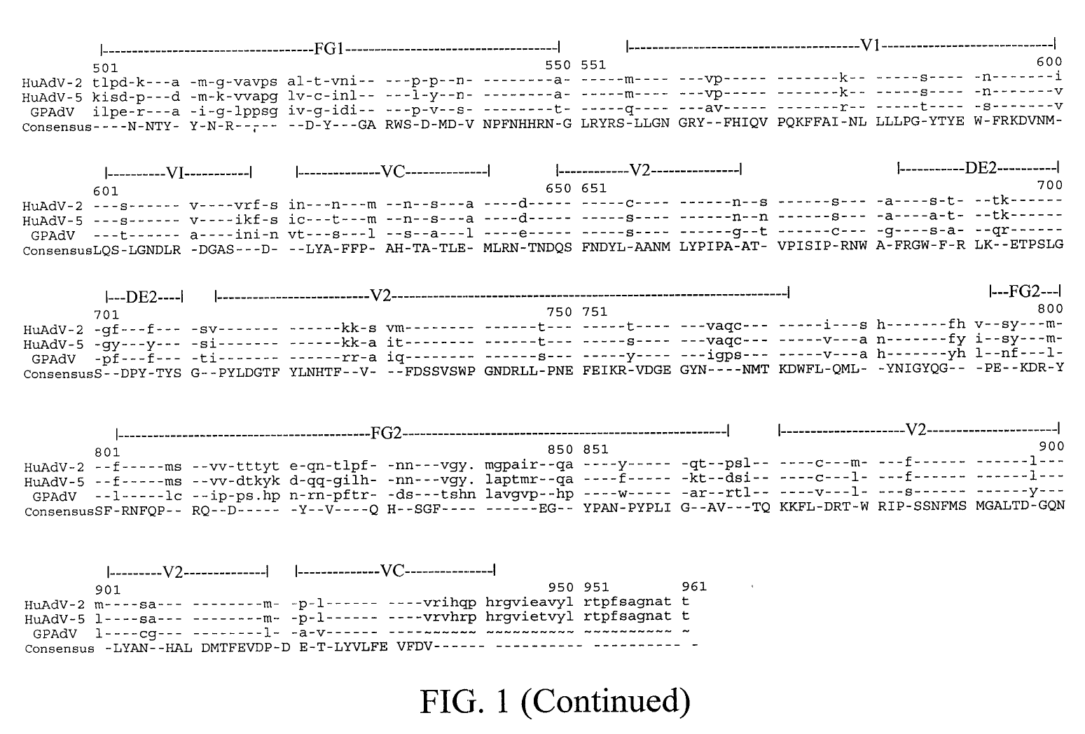

Hexon amino acid sequence alignment of GPAdV, HuAdV2 and HuAdV5 are shown in FIG. 1. X-ray crystallographic analysis of the structure of hexon protein has been reported for these two human adenoviruses. The deduced GPAdV hexon amino acid sequence corresponds to those portions of the human viral proteins designated NT, V1, V2, DE1, DE2, FG1, FG2 and ⅓ of VC in these two human adenoviruses (FIG. 1). The percentage of amino acid conservation by functional region has been determined by comparing the alignments of these GPAdV and HuAdV5. Regions V1 and V2 show the greatest percentage of amino acid conservation between (92% and 85%, respectively). In contrast, regions DE1, FG1 and FG2 have substantially less amino acid sequence conservation (27%, 55% and 50%, respectively). These latter three regions, along with region DE2, comprise the antigenic determinants of the hexon protein. Accordingly these regions where there is little similarity between the three adenoviruses can be used as antigenic fragments for vaccine formulations or for raising GPAdV hexoncoat specific antibodies. The smallest antigenic determinant (DE2, 24 amino acids) demonstrates a moderate amount of amino acid conservation (71%) in this comparison of GPAdV and HuAdV5. The sequence of amino acids in the amino terminal (NT) and carboxy terminal (VC) are moderately to highly conserved between these viruses (71% and 86%, respectively). Not all of the VC region could be analyzed, as it was not cloned in entirety using the above amplification strategy.

The most parsimonious dendrogram derived by analysis of the 18 hexon amino acid sequences was prepared with bootstrap support derived from 1000 bootstrap replications. All branch points derived from bootstrap analysis had a consensus greater than 500 of the 1000 datasets analyzed, with the exception of the branching of PorAdV3 (461 of 1000 dendrograms). GPAdV is phylogenetically most closely related to BovAdV3 (744 of 1000 dendrograms) in this comparison of hexon amino acid sequences. These two mastadenoviruses form a significant branch (778 of 1000 dendrograms) at a point that segregates the human adenoviruses and animal adenoviruses analyzed in this study. This method of hexon amino acid phylogenetic analysis related correctly the following groupings: HuAdV2 and 5 together (subgroup C, 1000 or 1000 dendrograms), HuAdV40 and 41 (subgroup F, 972 of 1000 dendrograms), HuAdV3 and 7 (subgroup B, 1000 of 1000 dendrograms). HuAdV12 (subgroup A, 997 of 1000 dendrograms) and HuAdV 48 (subgroup D, 937 of 1000 dendrograms) were correctly segregated from the other human adenoviruses analyzed. The similarity of CanAdV-1 and GPAdV hexon amino acid sequence is most likely a result of convergent evolution.

Discussion

The self-assembly of viral coat proteins is dependent on functional areas of protein-protein interaction. Peptides in the areas of coat protein involved in self-assembly likely contain amino acids of highly conserved sequence, as sequence variation would likely preclude capsid assembly and subsequent viral replication. Proceeding on this premise it was assumed there would be areas of conserved peptide sequence within the hexon and that this would convey to conservation of the underlying DNA gene sequence. Alignment of hexon gene sequences from several mastadenoviruses identified 3′ regions of conserved DNA sequence that were subsequently used in the development of a single long PCR encompassing more than 50% of the GPAdV hexon gene. This method overcame the difficulty in studying GPAdV due to lack of a purified source of genomic nucleic acid.

The deduced amino acid sequence of the conserved 3′ region of the hexon gene, used to generate the PCR fragment, corresponds with a region near the carboxyl-end of the V1 “viral jelly roll”. The conserved segmented regions of nucleic acid sequence encoding VI flanked the nucleic acid sequences encoding the DE1 and FG1 regions of the hexon. The highly variable regions (HVRs 1 through 7, Rux et al., Molecular Therapy 1:18-30 (2000) of structural entities DE1 and FG1 are associated with adenovirus type-specific antigenic determinants.

The guinea pig adenovirus has proven difficult to study due to the lack of an in vitro method of propagating the virus. Investigators have attempted to use antigens derived from other mastadenoviruses to study the incidence and/or development of antibodies in guinea pigs inoculated with GPAdV. These investigations have reported a lack of cross-reactivity, or weak cross-reactivity, of guinea pig convalescent sera when examined with heterotypic antigens. Lack of homotypic viral antigen for use in type specific antibody detection further hampered these studies. Antigen derived from MAD 1 and 2 (K87) have been used most often in attempts to detect antibodies to GPAdV. Convalescent serum antibodies of guinea pigs purposefully infected with GPAdV recognized MAD 1 and 2 antigens by the indirect fluorescent antibody method only when studied at low (or no) dilution. There is only a 29.22% similarity in amino acid sequence between GPAdV and MAD 1 in the region investigated, and thus a somewhat better antigenic preparation for detection of anti-GPAdV antibodies might be human adenovirus types 8 or 12. Most of the capsid mass of adenovirus is composed of three structural proteins, hexon, penton base and fiber. The surface of the icosahedral shaped adenoviruses is composed of 240 hexon molecules in 20 capsid faces. At the 12 vertices of the icosahedron are penton base molecules attached to fiber proteins, together referred to as penton. The hexon and penton proteins of adenovirus are the antigenic type-specific determinants of the capsid. Analyses of hexon residues with anti-peptide sera that can neutralize adenovirus infectivity in a type-specific manner suggest that the DE1 and FG1 regions are the antigenic determinants that are bound by these type-specific antibodies.

1. A purified polypeptide comprising the amino acid sequence of SEQ ID NO: 2, SEQ ID NO: 6, or an antigenic fragment of SEQ ID NO: 6.

2. The polypeptide of claim 1 wherein the polypeptide is immobilized on a solid support.

3. A composition comprising
an amino acid sequence selected from the group consisting of SEQ ID NO: 2, SEQ ID NO: 6, or an antigenic fragment of SEQ ID NO: 6; and
a pharmaceutically acceptable carrier.

4. The composition of claim 3, wherein the composition further comprises an adjuvant.

5. A purified nucleic acid sequence that hybridizes to SEQ ID NO: 7 or its complement under stringent conditions.

6. The nucleic acid sequence of claim 5 comprising a sequence selected from the group consisting of SEQ ID NO: 1 and SEQ ID NO: 5.

7. The nucleic acid sequence of claim 6 operably linked to regulatory elements capable of expressing the polypeptide.

8. An antibody that binds specifically to the protein of SEQ ID NO: 6.

9. A method for screening an animal for the presence of antibodies against GPAdV, said method comprising the steps of
contacting an antibody containing tissue sample from said animal with a hexon coat polypeptide;
removing non-specifically bound material;
detecting GPAdV antibodies that remain bound to the hexon coat polypeptide.

10. The method of claim 9 wherein the tissue sample is blood serum.

11. The method of claim 10 wherein the hexon coat polypeptide is immobilized on a solid surface and the step of removing the non-specifically bound material comprises washing the immobilized hexon coat polypeptide with a buffered solution.

12. The method of claim 11 wherein the step of detecting the GPAdV antibodies comprises contacting the immobilized hexon coat polypeptide with a labeled secondary antibody.

13. The method of claim 12 wherein the secondary antibody is labeled with a fluorophore or enzyme.

14. The method of claim 13 wherein the secondary antibody is labeled with an enzyme and the step of detecting the GPAdV antibodies comprises the use of an enzyme linked immunosorbent assay.

15. The method of claim 13 wherein the secondary antibody is labeled with a fluorophore and the step of detecting the GPAdV antibodies comprises the use of an indirect fluorescent immuno assay.

16. The method of claim 10 wherein the hexon coat polypeptide is selected from the group consisting of SEQ ID NO: 2, SEQ ID NO: 6, or an antigenic fragment of SEQ ID NO: 6.

17. The method of claim 16 wherein the hexon coat polypeptide is SEQ ID NO: 2.
